(19) United States
(12) Patent Application Publication
(10) Pub. No.: US 2011/0256075 A1
(43) Pub. Date: Oct. 20, 2011

(54) Cosmetic Composition Providing a Matte Effect, Process for Preparing Ucuhuba Butter and Use Thereof

(76) Inventors: Amanda Fernandes de Oliveira Dias, Campinas-SP (BR); Fabio Emanuel Bezerra Braga, Belem- PA (BR); Roberta Roesler, Campinas-SP (BR); Débora Cristina Castellani, Jundiaf-SP (BR)

(21) Appl. No.: 13/056,537

(22) PCT Filed: Jul. 30, 2009
(86) PCT No.: PCT/BR2009/000226

§ 371 Date: Jun. 13, 2011

(30) Foreign Application Priority Data
Jul. 30, 2009 (FR) 08/04341

(51) Int. Cl. A61K 8/97; A61Q 19/00; A61Q 1/06; A61Q 17/04; A61Q 1/02

(52) U.S. Cl. 424/59; 424/769; 424/63

(57) ABSTRACT

The present application relates to cosmetic compositions comprising ucuhuba butter (Virola surinamensis) capable of providing a matte effect, that is, eliminating the skin shine and/or oiliness. The present application also relates to the use of ucuhuba butter in body or facial emulsions and in makeup and to a process for the preparation of ucuhuba butter useful for the cosmetic compositions of the application.

### FIELD OF THE INVENTION

The present invention relates to cosmetic compositions comprising ucuhuba butter (Virola surinamensis) capable of providing a matte effect, that is, eliminating or reducing the skin shine and/or oiliness. The present invention also relates to the use of ucuhuba butter in body or facial emulsions and in makeup.

### DESCRIPTION OF THE PRIOR ART

In Brazil, the plant of the present invention is known as ucuúba, ucuúba-branca, ucuúba-cheirosa, ucuúba-de-igapó, ucuúba-da-várzea, ucuúba-verdadeira, bicuíba-branca, árvore-de-sebo, among other names. Ucuhuba belongs to the Myristicaceae family, of the Virola genus and surinamensis species.

The use of ucuhuba in the Amazon estuary region dates from a period before Brazilian colonization, when the natives used its seed and bark to manufacture hallucinogens in shamanic rituals. Since pre-Colombian times, the native Indians used some Virola species and called them “hiboucauhu”, “bicuda” and “ucuhuba”. “Ucuhuba”, in the native Tupi language, means “tree that produces an oily substance”; it comes from the words uku (fat, grease) and uba (tree). The Virola species are used in folk medicine in the cure of several diseases. In their travels, the Brazilian Indians used to take the sebum of the seeds for treating wounds.

The oil extracted from the seeds (ucuhuba sebum), which is rich in trimyristin and of pleasant smell, can be used in the manufacture of candles, soaps, cosmetics and perfumes. The sebum and sap have several applications in home medicine, especially in the treatment of rheumatism, arthritis, cramps, aphthae and hemorrhoids. Scientific studies are being conducted aiming at using its sebum in the treatment of malaria and Chagas disease. The tree provides abundant fruits for birds and other wild animals, thus being useful in recovering degraded and preservation areas.

From the 84 species of Myristicaceae found in the Americas, 59 are spread in the most diverse regions of Brazil, especially in the Amazon, the genus Virola, from the five native Brazilian Myristicaceae species, being the most representative one, being found in almost all the regions.

The species of the Virola genus are a fascinating field of research, given their importance as substance biosynthesizers, which has shown a great biological activity potential. In spite of this pharmacological aspect and the chemotaxonomic importance that provide unquestionable relevance to the Virola species, less than half of the species found in the Americas were studied to date, and most of these studies involve only the wood or the bark.

Among the classes of substances isolated from the species of the Virola genus, the tryptaminic and beta-carbonyl alkaloids are especially relevant, since they are responsible for the active ingredient of the snuff used by the Amazon Indians.

Neolignans are one of the major groups of substances occurring in the Myristicaceae family, also having important biological activity, such as, for instance, fungicidal activity.

Due to the presence of several active ingredients in the ucuhuba oil, its use in several products was broadly disseminated.

The inventions disclosed in patent documents U.S. Pat. No. 4,169,901, U.S. Pat. No. 5,047,166, U.S. Pat. No. 4,288,341, US 2006210505 and WO 2004/050108, for instance, show different applications of the ucuhuba oil or butter. However, none of the prior-art documents discloses the use of ucuhuba butter for achieving properties such as the matte effect, that is, the elimination of skin shine and/or oiliness.

Document U.S. Pat. No. 4,169,901 describes flavored deep-fat frying compositions. The compositions comprise: a base fat which is an edible triglyceride having acyl groups of form about 16 to 22 carbon atoms, wherein the smoke point exceeds about 350° F., and the Iodine Value is between from about 30 to about 150; a volatile artificial meat-like flavorant which is soluble and fat based; and a stabilizing agent which is an undeodorized edible oil selected from the group consisting of coconut oil, palm-kernel oil, cohune oil, muru muru oil, ucuhuba oil and babassu oil. The compositions are useful to impart meaty flavor to deep-fat fried foodstuffs for long periods of time.

Document U.S. Pat. No. 5,047,166 discloses a skin treatment composition comprising a physiologically acceptable carrier and 1-35% by weight of a salt of a monoester of citric acid. The skin treatment composition imparts a pleasant smoothness to the skin and may be incorporated in several products to treat skin dryness. The hydrophobic group having an ester linkage to citric acid has 10 to 18 carbon atoms. Monoester of citric acid may be synthesized with low levels of di- and triester by forming citric acid anhydride and reacting this to form the monoester, and the use of soaps having the fatty acid derived from ucuhuba butter is foreseen.

Document U.S. Pat. No. 4,288,341 discloses a deodorant toilet bar comprising a soap mixture; straight chain fatty acids and a deodorant composition. Said soap mixture can contain saponified ucuhuba butter. The soap can be employed for personal washing of the skin and will as a result reduce human body malodor.

Document US 2006210505 relates to multi-phase personal care compositions that comprise a first phase and a second phase, wherein said first and second phases form a visually distinct pattern. The compositions are intended for moisturizing or conditioning skin or hair and comprise less than about 10% by weight of the multiphase personal care composition and of surfactant. Ucuhuba butter is cited among the examples of waxes that can be added to said compositions.

Document WO 2004/050108 discloses and claims the use of oils obtained from plants of the Myristicaceae family in the preparation of dietetic, food, cosmetic, dermatological, pharmaceutical and veterinary compositions.

### OBJECTS OF THE INVENTION

The main object of the present invention is to provide cosmetic compositions comprising ucuhuba butter capable of conferring a matte effect (eliminating the skin shine and/or oiliness).

The present invention also relates to the use of ucuhuba butter in body or facial emulsions and in makeup.

### SUMMARY OF THE INVENTION

The present invention relates to a cosmetic composition capable of providing a matte effect comprising ucuhuba butter and one or more cosmetically acceptable adjuvants or carriers. Preferably, the ucuhuba butter is present in the composition at a concentration in the range of 0.5 to 50% by weight, particularly 5% by weight.

Examples of cosmetically acceptable adjuvants or carriers that can be used in the cosmetic composition according to the present invention comprise emollients, consistency agents, antioxidants, thickeners, preservatives, film-forming agents, sunscreens, coloring agents, fragrances, humectants, emulsifying agents and sensory agents.

Preferably, the emollients are selected among Ricinus communis (castor) seed oil, Brazilian nut oil, cetyl ricinoleate, caprylic/capric triglyceride, alkyl benzoate and octyl octanoate, and are present in the composition at a concentration in the range of 3 to 85% by weight.

Preferably, the consistency agents are selected among carnauba wax, candelilla wax, bee wax, sorbitan olivate, and are present in the composition at a concentration in the range of 1.5 to 17% by weight.

Particularly, the antioxidants are selected among oily vitamin E and butylated hydroxytoluene (BHT), and are present in the composition at a concentration in the range of 0.001 to 1.05% by weight.

Preferably, the thickeners are selected among cetyl alcohol and xanthan gum, and are present in the composition at a concentration in the range of 0.1 to 6% by weight.

Among the preservatives that may be used according to the present invention are: propylparaben, methylparaben, phenoxyethanol and potassium sorbate, being present in the composition at a concentration in the range of 0.1 to 1.1% by weight.

Preferably, the film-forming agent used according to the present invention is glycerin abietate at a concentration in the range of 1 to 3% by weight.

Sunscreens that can be used according to the present invention are, for instance, 2-ethyl-hexyl methoxycinamate, titanium dioxide and benzophenone-3, at a concentration in the range of 1.5 to 12% by weight.

Examples of coloring agents that can be used in the cosmetic composition of the present invention are cosmetically acceptable pigments and mica at a concentration in the range of 1 to 30% by weight.

An example of humectant that can be used in the cosmetic composition of the present invention is glycerin, which can be present in the composition at a concentration in the range of 1 to 3% by weight.

Particularly, the emulsifying agents are selected among sorbitan olivate and cetearyl olivate, and are present in the composition at a concentration in the range of 1 to 5% by weight.

Examples of sensory agents that can be used in the cosmetic composition of the present invention are mica and tapioca starch at a concentration in the range of 0.5 to 30% by weight.

The cosmetic compositions of the present invention can be in the form of body or facial emulsions or in the form of makeup, such as, for instance, in the form of a lipstick, foundation (fond de teint) or concealer (cacheceme).

The present invention also relates to the use of ucuhuba butter in the preparation of a cosmetic composition providing a matte effect. The ucuhuba butter can act as a matte effect agent, consistency agent, thickener, film-forming agent and/or emollient.

### DETAILED DESCRIPTION OF THE INVENTION

The present invention relates to cosmetic compositions comprising ucuhuba butter (Virola surinamensis) capable of providing a matte effect, that is, eliminating the skin shine and/or oiliness. The present invention also relates to the use of ucuhuba butter in body or facial emulsions and in makeup.

The ucuhuba butter in question suffers a purely physical process, which can be considered a “green technology”, that is, it contributes to environmental protection and does not cause byproducts or residues in the ucuhuba butter, as is the case, for instance, with chemical processes, such as solvent extraction, strong base neutralization etc.

The main steps in the processing of ucuhuba fruits are:

a) selecting fruits according to their ripening point (ripe);

b) drying the fruits by sun exposure and manual removal of seeds containing the pulp;

c) drying of seeds by sun exposure or in a greenhouse with forced air circulation—the final humidity should be from 7 to 9% so that they can be stored without contamination or germination risk.

d) cooking the seeds at 80 to 100° C. in a cooker to facilitate the release of the butter, which has a high melting point;

e) physical pressing of the seeds;

f) filtering the butter with the addition of a filtering agent;

g) treating the butter with organic acid and acidly activated clay by a certain period of time under vigorous stirring;

h) clarifying the butter for a certain period of time under vacuum at a certain temperature;

i) filtering under vacuum at a certain temperature; and

j) adding a scavenger and an antioxidant in an amount that is sufficient to obtain the final ucuhuba butter.

Currently, there is a huge interest in vegetable raw materials obtained exclusively by physical processes which may replace cosmetic raw materials that are manufactured from nonrenewable resources and by chemical processes.

Table 1 below shows the physical-chemical characterization of the clarified ucuhuba butter:

TABLE 1
Physical-Chemical Data

| | |
|---|---|
| Aspect | Solid |
| Color | Orange-colored |
| Odor | Characteristic |
| I. Saponification (mg KOH/g) | 225 to 238 |
| I. Iodine (g I₂/100 g) | 3 to 11 |
| Free Fatty Acids (% oleic) | max 10 |
| I. Peroxides (meq/Kg) | max 10 |

Fatty Composition (%)

| | |
|---|---|
| C8:0 (caprylic) | 0.1 |
| C10:0 (capric) | 0.6 |
| C12:0 (lauric) | 14.4 |
| C14:0 (myristic) | 71.6 |
| C16:0 (palmitic) | 5.4 |
| C16:1 (palmitoleic) | 0.4 |
| C18:0 (stearic) | 0.8 |
| C18:1 (oleic) | 5.7 |
| C18:2 (linoleic) | 0.5 |

Table 2 below shows the triglyceride composition of the ucuhuba butter:

TABLE 2

| Triglyceride | | |
|---|---|---|
| C36:0 | CCP | 0.29 |
| | LaLaLa | 0.44 |
| C38:0 | LaLaM | 4.60 |
| C40:0 | LaMM | 35.89 |
| | CMP | 0.10 |
| C42:0 | MMM | 42.50 |
| | LaMP | 1.84 |
| C44:0 | MMP | 5.62 |
| | LaPP | 2.16 |
| | LaMS | 0.45 |
| C44:1 | LaMO | 0.16 |
| C46:0 | MMS | 0.78 |
| C46:1 | MMO | 2.73 |
| | LaPO | 0.54 |
| C48:1 | MPO | 1.29 |
| C50:2 | MOO | 0.61 |

Fatty acids notation:
C—capric;
L—lauric;
M—myristic;
P—palmitic;
S—stearic;
O—oleic

In the cosmetic composition according to the present invention, in addition to the ucuhuba butter, several cosmetically acceptable adjuvants and carriers can be used, such as: emollients, consistency agents, antioxidants, thickeners, preservatives, film-forming agents, sunscreens, coloring agents, fragrances, humectants, emulsifying agents and sensory agents. It should be noted that other cosmetically acceptable adjuvants and carriers can also be used according to the present invention, depending on the cosmetic form to be prepared and/or the desired cosmetic/aesthetic effect.

### BRIEF DESCRIPTION OF THE DRAWINGS

FIG. 1 depicts the physical characterization of the ucuhuba butter, showing its melting curve. By the melting curve, it is possible to obtain the material melting range, as well as the temperature in which there is maximum melting (melting range: 29-48° C.).

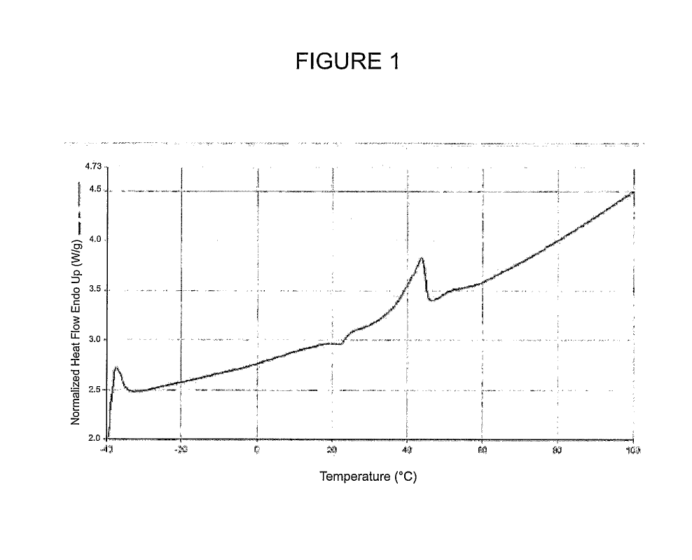

FIG. 2 is a graph assessing the sensory profile of ucuhuba butter and other cosmetically acceptable oils and butters.

FIG. 3 is a graph assessing the sensory profile of formulations containing ucuhuba butter, as well as products available in the market for oily skin.

FIG. 4 is a graph assessing the sensory profile of ucuhuba butter in comparison with cetyl lactate ester.

### TESTS FOR ANALYZING FORMULATIONS CONTAINING UCUHUBA BUTTER

By means of tests with a trained panel and sebumetry tests, it was possible to determine that compositions comprising ucuhuba butter has oiliness reduction and shine reduction attributes when said ucuhuba butter is present at certain concentrations in emulsion.

Efficacy and benefit mapping tests were performed with cosmetic compositions containing other cosmetically acceptable vegetable oils and butters but they did not show the results obtained with ucuhuba butter. Cosmetic compositions containing ucuhuba butter in accordance with the present invention were able effectively to reduce skin oiliness and shine. To the contrary, compositions with other cosmetically acceptable oils and butters strongly contribute to the oiliness and shine attributes, as widely discussed in the literature.

a) Trained Panel

Formulation 1:

The following composition was prepared containing the ucuhuba butter obtained according to the process described above at the ratio of 3% by weight:

| Component | Concentration (% w/w) |
|---|---|
| demineralized water | 92.15 |
| disodium EDTA | 0.05 |
| carbopol ETD 2020 | 0.45 |
| Triethanolamine | 0.45 |
| ucuhuba butter | 3.00 |
| iodopropynyl butylcarbamate | 0.20 |
| phenoxyethanol F | 0.70 |
| hydroxyethyl acrylate, | 3.00 |
| acryloyldimethyl copolymer | |

The composition above was obtained by initially preparing the aqueous phase adding EDTA to the water and waiting its total solubilization. Then, Carbopol was gradually added until its total dispersion, following by the addition of triethanolamine. For the preparation of the oily phase, the ucuhuba butter was heated up to its melting point. After heating the aqueous phase up to the same temperature of the ucuhuba butter, the two phases were mixed and stirred for 5 minutes. The heating ceased, the other components were added and stirring continued for additional 15 minutes.

Formulation 2:

The following composition was prepared containing the ucuhuba butter obtained according to the process described for Formulation 1 at the ratio of 5% by weight:

| Component | Concentration (% w/w) |
|---|---|
| demineralized water | 90.15 |
| disodium EDTA | 0.05 |
| carbopol ETD 2020 | 0.45 |
| triethanolamine | 0.45 |
| ucuhuba butter | 5.00 |
| iodopropynyl butylcarbamate | 0.20 |
| phenoxyethanol F | 0.70 |
| hydroxyethyl acrylate, | 3.00 |
| acryloyldimethyl copolymer | |

Formulation 3:

The following composition was prepared containing the ucuhuba butter obtained according to the process described for Formulation 1 at the ratio of 10% by weight:

| Component | Concentration (% w/w) |
|---|---|
| demineralized water | 85.15 |
| disodium EDTA | 0.05 |
| carbopol ETD 2020 | 0.45 |
| triethanolamine | 0.45 |
| ucuhuba butter | 10.00 |
| iodopropynyl butylcarbamate | 0.20 |
| phenoxyethanol F | 0.70 |
| hydroxyethyl acrylate, | 3.00 |
| acryloyldimethyl copolymer | |

Formulation 4:

The following composition was prepared, called placebo, obtained according to the process described for Formulation 1:

| Component | Concentration (% w/w) |
|---|---|
| demineralized water | 90.15 |
| disodium EDTA | 0.05 |
| carbopol ETD 2020 | 0.45 |
| triethanolamine | 0.45 |
| ucuhuba butter | 5.00 |
| iodopropynyl butylcarbamate | 0.20 |
| phenoxyethanol F | 0.70 |
| hydroxyethyl acrylate, | 3.00 |
| acryloyldimethyl copolymer | |

Objective:

Identify and compare the sensory profile of formulations 1 to 4 with compositions containing other cosmetically acceptable vegetable and mineral oils and butters.

Methodology:

The panelists assessed 12 sensory attributes listed in Table 3 using linear scales of 10 points and quantitative references.

The products have been assessed randomly in three repetitions each.

TABLE 3
Description of attributes assessed in the study with trained
panel:

| Attribute | Definition |
|---|---|
| Absorption Point | Number of rotations needed for the |
| | product to start being absorbed by |
| | the skin |
| Spreadability | Easiness to spread the product on |
| | the skin |
| Slipperiness | Easiness to slide/move the finger on |
| | the skin |
| Immediate skin gloss | Light intensity reflected on the skin |
| | immediately after the product is spread |
| Residual skin gloss | Light intensity reflected on the skin |
| | two minutes after the product is spread |
| Stickiness | Intensity with which the finger adheres |
| | to the skin |
| Immediate oiliness | Oily sensation on the skin during and |
| | after the product is spread |
| Residual oiliness | Oily sensation on the skin 2 minutes |
| | after the product is spread |
| Immediate greasy film | Greasy sensation forming a film on |
| | the skin immediately after the product |
| | is spread |
| Residual greasy film | Greasy sensation forming a film on |
| | the skin 2 minutes after the product is |
| | spread |
| Velvety film | Peach skin sensation |
| Residual whiteness | Formation of a white film on the skin |

The products were assessed in 5 cm wide circular sites, in the forearm region, two fingers away from the wrists and the elbows. The amount of product applied in each site was 25 μL, spread in circles, obeying the rhythm of a metronome, at the rate of 120 pulses per minute.

### Products Assessed

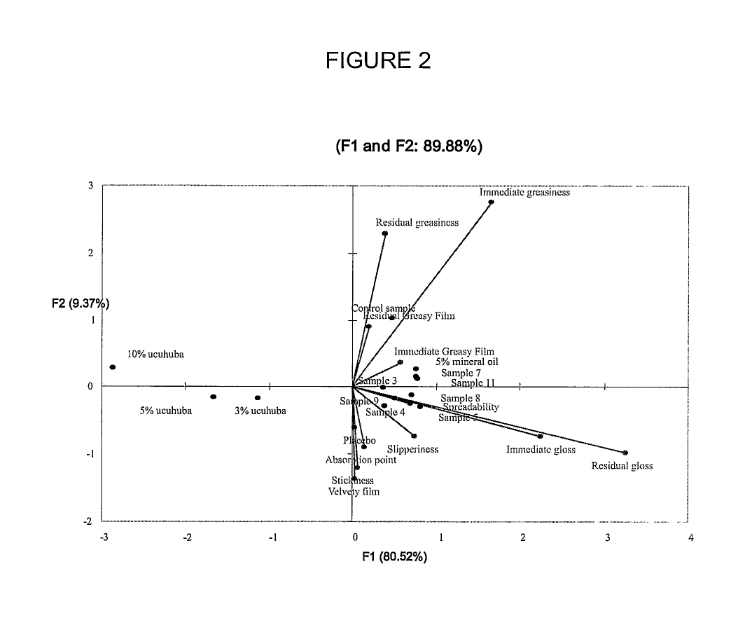

Butters/oils were applied in a wash out emulsion at the concentrations of 3%, 5% and 10% by weight. The products assessed are listed in Table 4 below and the results are shown in FIG. 2.

TABLE 4

| Product | Concentration (% by weight) |
|---|---|
| Ucuhuba butter 10% | 10% |
| Ucuhuba butter 3% | 3% |
| Vegetable butter (Sample 3) | 5% |
| Vegetable butter (Sample 4) | 5% |
| Vegetable butter (Sample 5) | 5% |
| Ucuhuba butter 5% | 5% |
| Vegetable oil (Sample 7) | 5% |
| Vegetable oil (Sample 8) | 5% |
| Vegetable oil (Sample 9) | 5% |
| Mineral oil 5% | 5% |
| Vegetable oil (Sample 11) | 5% |
| Placebo | — |
| Control sample | — |

By means of the multivariate analysis of the attributes that characterize a product, it is noted by the results obtained (FIG. 2) that the emulsions containing ucuhuba butter in different concentrations have a very differentiated sensory profile and are consequently located in the sensory map (FIG. 2) in completely opposite axes in relation to the oil and butter emulsions commonly employed for cosmetic applications, characterized by the attributes of gloss, oiliness and greasy film. In addition, it is noted that an increase in the concentration of the ucuhuba butter in emulsion intensifies the sensory attributes of gloss and oiliness reduction, that is to say, the increase of the ucuhuba butter in the emulsion causes the reduction of the attributes that are characteristics of an oily skin, rendering the application of ucuhuba butter in emulsions and makeup advantageous for oily skins.

Furthermore, ucuhuba butter can be used as a consistency agent and thickener in cosmetic formulations in view of the attributes of spreadability, slipperiness and high melting point.

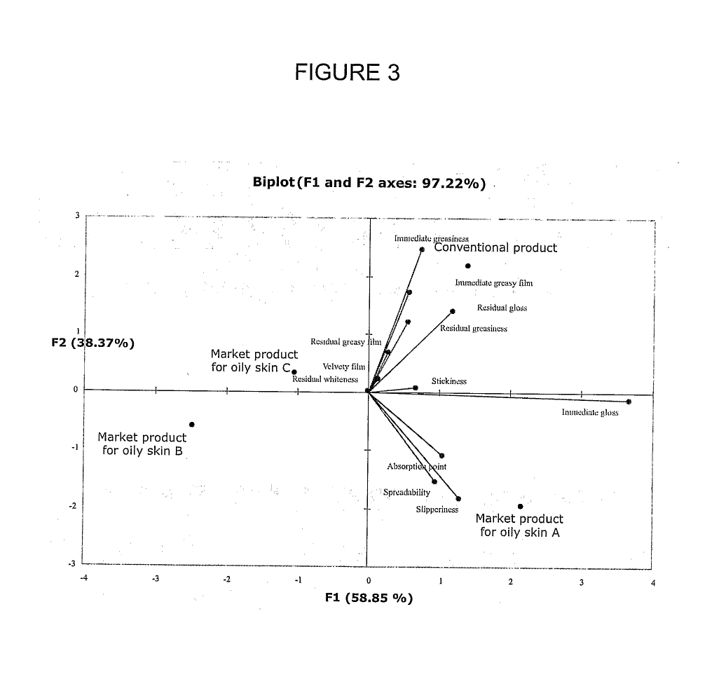

The products for oily skin available in the market were also investigated by multivariate analysis of sensory data, and it was proven that their location in the sensory map (FIG. 3) is similar to or better than that of the products formulated with ucuhuba butter (FIG. 2), that is, opposite to the vectors that characterize the attributes of oiliness and gloss.

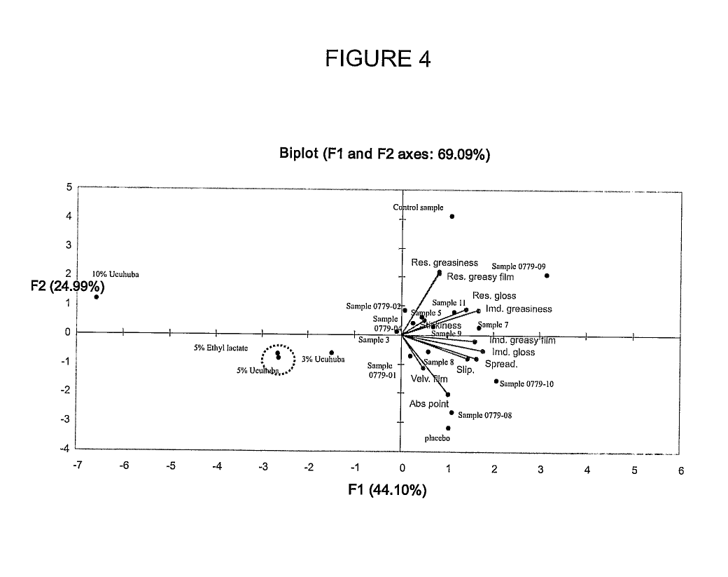

Finally, the same technique of multivariate sensory analysis was used to compare the emulsions containing ucuhuba butter with plant esters widely used in cosmetics. By means of the results of the sensory map (FIG. 4), it is noted that ucuhuba butter has a very similar profile to that of the cetyl lactate ester. The use of this ester in cosmetic formulations implies the inclusion of a warning in the label of the product in some countries, such as the United States. Therefore, ucuhuba butter can serve as a totally vegetable and natural substitute for cetyl lactate ester in cosmetic formulations, and can be used without any type of restriction.

b) Sebumetry

For the sebumetry test, the same composition described in Formulation 2 was prepared (ucuhuba butter 5%):

| Component | Concentration (% w/w) |
|---|---|
| demineralized water | 95.15 |
| disodium EDTA | 0.05 |
| carbopol ETD 2020 | 0.45 |
| triethanolamine | 0.45 |
| iodopropynyl butylcarbamate | 0.20 |
| phenoxyethanol F | 0.70 |
| hydroxyethyl acrylate, | 3.00 |
| acryloyldimethyl copolymer | |

Objective:

To assess the evolution of skin oiliness in time, using the sebumetry technique and comparing treated and untreated sites.

Description:

Measurement of the oiliness of skin, hair and scalp using MPA 5-Sebumeter is based on the grease-spot photometry method, which is not influenced by humidity. Sebum is collected on a translucent plastic tape of the sebumeter cassette which becomes transparent, and the result is obtained by measuring the difference in the light transmittance value through the tape, before and after impregnation with sebum.

Methodology:

Twenty women were selected ranging between 20 and 50 years old, in conformity with the inclusion criteria. The volunteers were instructed to stop using any cosmetic products in their forearms 48 hours before the beginning of the tests. The volunteer remained in acclimatization for 15 minutes at 22±2° C. and 55±5% relative humidity. In the forehead of the volunteers, over the central region of each eyebrow, a 2×2 cm site was marked so as to keep the right and left sites aligned in the horizontal direction. Randomly, the sample under study or the standard were applied to one of the sites, keeping it under pressure for 30 seconds according to the instructions guide. Readings were carried out for the right and left sites, the so-called baseline t0 reading. Randomly, the sample under study or the standard were applied to one of the sites, keeping the other as control, without applying any product. 20 μL of product were applied, or 5 μL/cm². The material was evenly spread with the index finger using a disposable finger pad. The volunteer waited 15 minutes for drying. After drying, half of a cotton disk was soaked with 1.0 mL of distilled water and the product was removed with three passes in the same sense, while keeping constant pressure. The same removal procedure with cotton and water was carried out for the control site, which was not treated (a pilot was carried out in advance to see if this removal would be necessary, and whether or not the product would interfere). After 15 minutes of drying, a new oiliness reading was carried out in the treated and control sites, called t15. The readings were repeated at times 1, 2, 3 and 4 h after application.

Tested Products:

Ucuhuba butter 5% by weight

Sample A (cosmetically acceptable vegetable oil)

Sample B (cosmetically acceptable vegetable butter)

Placebo

Control

Results:

The results as a function of time in relation to placebo and control are presented in Table 5 below:

TABLE 5

| | Product vs. Control | | | | | Product vs. Placebo | | | | | |
|---|---|---|---|---|---|---|---|---|---|---|---|
| | min | | | | | min | | | | | |
| Product | 15 | 60 | 120 | 180 | 240 | 15 | 60 | 120 | 180 | 240 | |
| Ucuhuba | X | X | X | | | | X | | | | Product |
| butter 5% | | | | | | | | | | | better than |
| | | | | | | | | | | | placebo |
| Placebo | X | X | | | | | | | | | |
| Sample A | | X | X | X | | | | X | X | | Placebo |
| | | | | | | | | | | | better than |
| | | | | | | | | | | | product |
| Sample B | | X | | | | | | | | X | Placebo |
| | | | | | | | | | | | better than |
| | | | | | | | | | | | product |

The products were assessed and compared with the placebo and the control. The results show that ucuhuba butter at 5% in a wash out emulsion reduces skin oiliness in 15 minutes, 1 hour and 2 hours after its application when compared with the control and reduces skin oiliness in 1 hour when compared with the placebo. Samples A and B (containing other oils and butters commonly used in cosmetic formulations) showed inferior results when compared to the placebo. Therefore, such results were much lower than those obtained by the emulsion containing ucuhuba butter at 5% by weight. By means of the sebumetry test, it is noted that the ucuhuba butter in emulsion confers a quantitative reduction of the skin oiliness for 15 minutes, 1 hour and 2 hours, in addition to the sensory attributes of gloss and oiliness reduction seen in the sensory map.

### EXAMPLES OF EMBODIMENTS

The cosmetic compositions according to the present invention can be in the form of body or facial emulsions or makeup. For illustrative purposes, below are some examples of embodiments of the cosmetic composition containing ucuhuba butter providing a matte effect according to the present invention.

A lipstick, a foundation and a concealer were prepared containing ucuhuba butter according to the usual techniques used in cosmetics. In the cosmetic composition according to the present invention, in addition to ucuhuba butter, several cosmetically acceptable adjuvants and carriers can be used, such as: emollients, consistency agents, antioxidants, thickeners, preservatives, film-forming agents, sunscreens, coloring agents, fragrances, humectants, emulsifying agents and sensory agents. It should be noted that other cosmetically acceptable adjuvants and carriers can also be used according to the present invention, depending on the cosmetic form to be prepared and/or the desired cosmetic/esthetic effect.

### Example 1

### Lipstick

The following lipstick composition containing ucuhuba butter was prepared at the ratio of 0.5 to 50% by weight:

| | | Preferred | |
|---|---|---|---|
| | Concentration | Concentration | |
| Component | (% by weight) | (% by weight) | Function |
| Ricinus communis | 20.00-40.00 | 20.0 | emollient |
| (castor) seed oil | | | |
| Ucuhuba butter | 0.50-50.00 | 5.0 | emollient, |
| | | | consistency agent, |
| | | | matte effect |
| Carnauba wax | 0.50-5.00 | 1.0 | consistency agent |
| Candelilla wax | 0.50-5.00 | 5.0 | consistency agent |
| Bee wax | 0.50-7.00 | 1.0 | consistency agent |
| Brazilian nut oil | 5.00-20.00 | 19.25 | emollient |
| Cetyl ricinoleate | 5.00-15.00 | 2.5 | emollient |
| Oily Vitamin E | 0.1-1.00 | 0.5 | antioxidant |
| Cetyl alcohol | 2.00-6.00 | 3.0 | thickener |
| Propylparaben | 0.05-0.10 | 0.1 | preservative |
| Methylparaben | 0.05-0.20 | 0.2 | preservative |
| Butylated | 0.01-0.05 | 0.05 | antioxidant |
| hydroxytoluene | | | |
| Caprylic/Capric | 10.00-20.00 | 20.0 | emollient |
| triglyceride | | | |
| Glyceryl abietate | 1.00-3.00 | 2.0 | film-forming agent |
| 2-ethyl-hexyl | 1.00-7.00 | 5.0 | sunscreen |
| methoxycinamate | | | |
| Titanium dioxide | 1.00-3.00 | 2.4 | sunscreen |
| Benzophenone-3 | 0.50-2.00 | 1.0 | sunscreen |
| Pigment | 0.50-10.00 | 6.0 | coloring agent |
| Mica | 0.50-10.00 | 5.0 | coloring agent |
| Aroma | 0.20-1.00 | 1.0 | fragrance |

The cosmetic composition described above (lipstick) provides a matte effect as disclosed in the present application.

### Example 2

### Foundation

The following base composition containing ucuhuba butter was prepared at the ratio of 0.5 to 50% by weight:

| | | Preferred | |
|---|---|---|---|
| | Concentration | Concentration | |
| Component | (% by weight) | (% by weight) | Function |
| Demineralized | q.s.p | 63.85 | carrier |
| water | | | |
| Glycerin | 1.00-3.00 | 2.00 | humectant |
| Xanthan gum | 0.10-0.80 | 0.50 | thickener |
| Alkyl benzoate | 2.00-6.00 | 5.0 | emollient |
| Sorbitan olivate | 0.50-2.00 | 1.5 | emulsifying agent |
| 2-ethyl-hexyl | 1.00-7.00 | 3.0 | sunscreen |
| methoxycinamate | | | |
| Benzophenone-3 | 0.50-2.00 | 1.0 | sunscreen |
| Ucuhuba butter | 0.50-50.00 | 2.0 | consistency agent, |
| | | | matte effect, |
| | | | film-forming agent |
| Capric/caprylic | 1.00-5.00 | 4.0 | emollient |
| triglyceride | | | |
| Cetearyl olivate | 0.50-3.00 | 3.0 | emulsifying agent |
| B.H.T. | 0.01-0.05 | 0.05 | antioxidant |
| Tapioca starch | 0.50-4.00 | 3.0 | sensory agent |
| Pigment | 3.00-15.00 | 10.0 | coloring agent |
| Phenoxyethanol | 0.1-1.00 | 0.9 | preservative |
| Potassium sorbate | 0.05-0.10 | 0.1 | preservative |
| Aroma | 0.10-0.50 | 0.1 | fragrance |

The cosmetic composition described above (foundation) provides a matte effect as disclosed in the present application.

### Example 3

### Concealer

The following corrective composition containing ucuhuba butter was prepared at the ratio of 1 to 50% by weight:

| | | Preferred | |
|---|---|---|---|
| | Concentration | Concentration | |
| Component | (% by weight) | (% by weight) | Function |
| Octyl octanoate | 5.00-10.00 | 10.0 | emollient |
| Capric/caprylic | 10.00-20.00 | 15.0 | emollient |
| triglyceride | | | |
| Ricinus communis | 20.00-30.00 | 27.95 | emollient |
| (castor) seed oil | | | |
| Pigment | 5.00-20.00 | 20.0 | coloring agent |
| Tapioca starch | 5.00-20.00 | 15.0 | sensory agent |
| Ucuhuba butter | 1.00-50.00 | 3.0 | consistency agent, |
| | | | matte effect |
| Carnauba wax | 0.50-5.00 | 0.5 | consistency agent |
| Candelilla wax | 0.50-5.00 | 4.0 | consistency agent |
| Sorbitan olivate | 1.00-3.00 | 3.0 | consistency agent |
| B.H.T. | 0.01-0.05 | 0.05 | antioxidant |
| Mica | 1.00-10.00 | 1.50 | coloring agent, |
| | | | sensory agent |

The cosmetic composition described above (concealer) provides a matte effect as disclosed in the present application.

The present invention has been described based on 3 examples of embodiments; it is understood, however, that it encompasses other embodiments and is limited only by the scope of the appended claims.

1. A cosmetic composition providing a matte effect, characterized by comprising ucuhuba butter and a cosmetically acceptable adjuvant or carrier.

2. A composition according to claim 1, characterized by comprising 0.5 to 50% by weight of ucuhuba butter.

3. A composition according to claim 2, characterized by comprising 5% by weight of ucuhuba butter.

4. A composition according to any one of claims 1 to 3, characterized in that the cosmetically acceptable adjuvant or carrier comprises one or more among emollients, consistency agents, antioxidants, thickeners, preservatives, film-forming agents, sunscreens, coloring agents, fragrances, humectants, emulsifying agents and sensory agents.

5. A composition according to claim 4, characterized in that the emollients are selected among Ricinus communis (castor) seed oil, Brazilian nut oil, cetyl ricinoleate, caprylic/capric triglyceride, alkyl benzoate and octyl octanoate at a concentration in the range of 3 to 95% by weight.

6. A composition according to claim 4, characterized in that the consistency agents are selected among carnauba wax, candelilla wax, bee wax, and sorbitan olivate at a concentration in the range of 1.5 to 17% by weight.

7. A composition according to claim 4, characterized in that anti-oxidants are selected among oily vitamin E and butylated hydroxytoluene (BHT) at a concentration in the range of 0.001 to 1.05% by weight.

8. A composition according to claim 4, characterized in that the thickeners are selected among cetyl alcohol and xanthan gum at a concentration in the range of 0.1 to 6% by weight.

9. A composition according to claim 4, characterized in that the preservatives are selected among propylparaben, methylparaben, phenoxyethanol and potassium sorbate at a concentration in the range of 0.1 to 1.1% by weight.

10. A composition according to claim 4, characterized in that the film-forming agent is glycerin abietate at a concentration in the range of 1 to 3% by weight.

11. A composition according to claim 4, characterized in that the sunscreens are selected among 2-ethyl-hexyl methoxycinamate, titanium dioxide and benzophenone-3 at a concentration in the range of 1.5 to 12% by weight.

12. A composition according to claim 4, characterized in that the coloring agents are selected among cosmetically acceptable pigments and mica at a concentration in the range of 1 to 30% by weight.

13. A composition according to claim 4, characterized in that the humectant is glycerin at a concentration in the range of 1 to 3% by weight.

14. A composition according to claim 4, characterized in that the emulsifying agents are selected among sorbitan olivate and cetearyl olivate at a concentration in the range of 1 to 5% by weight.

15. A composition according to claim 4, characterized in that the sensory agents are selected among mica and tapioca starch at a concentration in the range of 0.5 to 30% by weight.

16. A composition according to any one of claims 1 to 15, characterized by being in the form of body or facial emulsions or in the form of makeup, such as lipstick, foundation or concealer.

17. Use of ucuhuba butter, characterized by being in the preparation of a cosmetic composition providing a matte effect.

18. Use according to claim 17, characterized in that the ucuhuba butter acts as a matte effect agent, consistency agent, thickener, film-forming agent and/or emollient.

19. Use according to claim 17 or 18, characterized in that the cosmetic composition is in the form of body or facial emulsions or in the form of makeup, such as lipstick, foundation or concealer.

20. Process for preparing ucuhuba butter characterized in that it comprises the steps of:
a) selecting ripe ucuhuba fruits;
b) drying the ripe fruits by sun exposure and manually removing the seeds containing the pulp;
c) drying the seeds obtained in step (b) until a final humidity of from 7 to 9%;
d) physically pressing of the seeds to obtain the butter;
e) filtering the butter obtained in (d) with the addition of a filtering agent;
f) treating the butter with organic acid and acidly activated clay under vigorous stirring;
g) clarifying the butter using vacuum;
h) filtering the clarified butter under vacuum; and
i) adding a scavenger and an antioxidant to the filtered butter obtained in (h) to obtain the final ucuhuba butter.

21. Process according to claim 20, characterized by comprising a step of cooking the seeds in a temperature in the range of 80 to 100° C. prior to the pressing step (d).
